(19) United States
(12) Patent Application Publication
(10) Pub. No.: US 2009/0192617 A1
(43) Pub. Date: Jul. 30, 2009

(54) Intervertebral Prosthetic Disc With Shock Absorbing Core Formed With Disc Springs

(75) Inventors: Yves Arramon, Sunnyvale, CA (US); Malan de Villiers, Wapadrand (ZA); Neville Jansen, Waterkloof (ZA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SpinalMotion, Inc., Mountain View, CA (US)

(21) Appl. No.: 12/358,716

(22) Filed: Jan. 23, 2009

Related U.S. Application Data
(60) Provisional application No. 61/023,480, filed on Jan. 25, 2008.
(60) Provisional application No. 61/049,259, filed on Apr. 30, 2008.

(51) Int. Cl. A61F 2/44

(52) U.S. Cl. 623/17.16; 623/17.13

(57) ABSTRACT

An artificial intervertebral disc has upper and lower prosthesis plates disposed about a shock absorbing mobile core. The shock absorbing core includes one or more spring washers or disc springs between rigid upper and lower surfaces of the core to allow the upper and lower surfaces to move resiliently toward and away from each other. This allows the core to absorb forces applied to it by the vertebrae. The components of the shock absorbing core including the disc springs are formed of rigid materials having high durability and biocompatibility.

### CROSS-REFERENCES TO RELATED APPLICATIONS

This application claims priority to U.S. Provisional Application No. 61/023,480 filed Jan. 25, 2008, entitled “INTERVERTEBRAL PROSTHETIC DISC WITH SHOCK ABSORBING CORE FORMED WITH DISC SPRINGS” and U.S. Provisional Application No. 61/049,259 filed Apr. 30, 2008, entitled “INTERVERTEBRAL PROSTHETIC DISC WITH SHOCK ABSORBING CORE FORMED WITH DISC SPRINGS;” the full disclosures of which are incorporated herein by reference.

### BACKGROUND OF THE INVENTION

The present invention relates to medical devices and methods. More specifically, the invention relates to intervertebral disc prostheses.

Back pain takes an enormous toll on the health and productivity of people around the world. According to the American Academy of Orthopedic Surgeons, approximately 80 percent of Americans will experience back pain at some time in their life. In the year 2000, approximately 26 million visits were made to physicians' offices due to back problems in the United States. On any one day, it is estimated that 5% of the working population in America is disabled by back pain.

One common cause of back pain is injury, degeneration and/or dysfunction of one or more intervertebral discs. Intervertebral discs are the soft tissue structures located between each of the thirty-three vertebral bones that make up the vertebral (spinal) column. Essentially, the discs allow the vertebrae to move relative to one another. The vertebral column and discs are vital anatomical structures, in that they form a central axis that supports the head and torso, allow for movement of the back, and protect the spinal cord, which passes through the vertebrae in proximity to the discs.

Discs often become damaged due to wear and tear or acute injury. For example, discs may bulge (herniate), tear, rupture, degenerate or the like. A bulging disc may press against the spinal cord or a nerve exiting the spinal cord, causing “radicular” pain (pain in one or more extremities caused by impingement of a nerve root). Degeneration or other damage to a disc may cause a loss of “disc height,” meaning that the natural space between two vertebrae decreases. Decreased disc height may cause a disc to bulge, facet loads to increase, two vertebrae to rub together in an unnatural way and/or increased pressure on certain parts of the vertebrae and/or nerve roots, thus causing pain. In general, chronic and acute damage to intervertebral discs is a common source of back related pain and loss of mobility.

When one or more damaged intervertebral discs cause a patient pain and discomfort, surgery is often required. Traditionally, surgical procedures for treating intervertebral discs have involved discectomy (partial or total removal of a disc), with or without fusion of the two vertebrae adjacent to the disc. Fusion of the two vertebrae is achieved by inserting bone graft material between the two vertebrae such that the two vertebrae and the graft material grow together. Oftentimes, pins, rods, screws, cages and/or the like are inserted between the vertebrae to act as support structures to hold the vertebrae and graft material in place while they permanently fuse together. Although fusion often treats the back pain, it reduces the patient's ability to move, because the back cannot bend or twist at the fused area. In addition, fusion increases stresses at adjacent levels of the spine, potentially accelerating degeneration of these discs.

In an attempt to treat disc related pain without fusion, an alternative approach has been developed, in which a movable, implantable, artificial intervertebral disc (or “disc prosthesis”) is inserted between two vertebrae. A number of different artificial intervertebral discs are currently being developed. For example, U.S. Patent Application Publication Nos. 2005-0021146, 2005-0021145, and 2006-0025862, which are hereby incorporated by reference in their entirety, describe artificial intervertebral discs. Other examples of intervertebral disc prostheses are the CHARITE artificial disc (provided by DePuy Spine, Inc.) MOBIDISC (provided by LDR Medical (www.ldrmedical.fr)), the BRYAN Cervical Disc (provided by Medtronic Sofamor Danek, Inc.), the PRODISC (from Synthes Stratec, Inc.), and the PCM disc (provided by Cervitech, Inc.). Although existing disc prostheses provide advantages over traditional treatment methods, improvements are ongoing.

The known artificial intervertebral discs generally include upper and lower plates or shells which locate against and engage the adjacent vertebral bodies, and a core for providing motion between the plates. The core may be movable or fixed, metallic, ceramic or polymer and generally has at least one convex outer surface which mates with a concave recess on one of the plate in a fixed core device or both of the plates for a movable core device such as described in U.S. Patent Application Publication No. 2006-0025862. However, currently available artificial intervertebral discs do not provide for cushioning or shock absorption which would help absorb forces applied to the prosthesis from the vertebrae to which they are attached. A natural disc is largely fluid which compresses to provide cushioning. It would be desirable to mimic some of this cushioning in an artificial disc.

Therefore, a need exists for an improved artificial intervertebral disc. Ideally, such improved disc would avoid at least some of the short comings of the present discs while providing at least some shock absorption.

### BRIEF SUMMARY OF THE INVENTION

Embodiments of the present invention provide an artificial intervertebral disc with shock absorption and methods of providing shock absorption with an artificial disc. The prosthesis system comprises supports that can be positioned against vertebrae and a shock absorbing core that can be positioned between the supports to allow the supports to articulate.

In a first aspect an artificial intervertebral disc includes upper and lower supports and a core positioned between the upper and lower supports. The core is movable with respect to the upper and lower supports. Each of the upper and lower supports includes an outer surface which engages a vertebra and an inner bearing surface. The core includes upper and lower core members configured to engage the inner bearing surfaces of the upper and lower support plates and at least one spring washer in the core to provide compliance to the core. The spring washer allows the upper and lower core members to move resiliently toward and away from each other.

According to another aspect of the invention, a method of assembling a compliant mobile core for an artificial intervertebral disc includes the steps of providing upper and lower core members; positioning at least one disc spring between the upper and lower core members in an arrangement which allows the upper and lower core members to move resiliently toward and away from each other; and locking the upper and lower core members together in a manner which traps the at least one disc spring in place between the upper and lower core members.

According to a further aspect of the invention, a mobile core for an artificial intervertebral disc includes upper and lower core members each having an outer bearing surface and an inner surface; at least one spring washer positioned between the inner surfaces of the upper and lower core members to allow the upper and lower core members to move resiliently toward and away from each other; and a retention feature on the upper and lower core members which prevents the spring washer from being removed from the core during use.

According to an additional aspect of the present invention, an artificial intervertebral disc includes upper and lower supports and a core positioned between the upper and lower supports and movable with respect to the upper and lower supports. Each support includes an outer surface which engages a vertebra and an inner bearing surface. The core includes upper and lower core members configured to engage the inner bearing surfaces of the upper and lower support plates and at least one disc spring in the core to provide compliance to the core which allows the upper and lower core members to move resiliently toward and away from each other.

### BRIEF DESCRIPTION OF THE DRAWINGS

FIG. 1 is a side cross sectional view of an artificial disc with a shock absorbing core including a plurality of curved spring washers;

FIG. 2 is a perspective view of the shock absorbing core of FIG. 1;

FIG. 3 is a perspective view of a shock absorbing core with flat washers;

FIG. 4 is a cross sectional view the shock absorbing core of FIG. 3 having flat washers;

FIG. 5A is a perspective view a flat washer used in the shock absorbing core of FIG. 3;

FIG. 5B is a cross sectional view the flat washer used in the shock absorbing core of FIG. 3;

FIG. 6 is a perspective view of a shock absorbing core with flat washers as in FIG. 3 rearranged in a combined parallel and series arrangement;

FIG. 7 is a cross sectional view the shock absorbing core of FIG. 6;

FIG. 8 is a perspective view of another example of a shock absorbing core having cone shaped springs;

FIG. 9 is a cross sectional view of the shock absorbing core of FIG. 8;

FIG. 10 is a perspective view of a shock absorbing core according to one embodiment of the present invention with wave washers;

FIG. 11 is a cross sectional view of the core of FIG. 10;

FIG. 12 is a perspective view of the wave spring of the core of FIG. 10;

FIG. 13 is a cross sectional view of a shock absorbing core with flat disc springs;

FIG. 14 is a perspective view of another shock absorbing core with a split ring spring washer;

FIG. 15 is a cross sectional view of the core of FIG. 14;

FIG. 16 is a perspective view of the split ring spring washer for the core of FIG. 14;

FIG. 17 is a perspective view of a further shock absorbing core with another version of a split ring spring washer;

FIG. 18 is a cross sectional view of the core of FIG. 17;

FIG. 19 is an exploded perspective view of an alternative embodiment of a core with a coil spring washer;

FIG. 20 is a cross sectional view of the assembled core of FIG. 19 in a compressed configuration; and

FIG. 21 is a cross sectional view of an alternative embodiment of a core with a coil spring washer and a locking feature.

### DETAILED DESCRIPTION OF THE INVENTION

Various embodiments of the present invention generally provide for an artificial intervertebral disc having upper and lower plates disposed about a shock absorbing mobile core. The mobile core design provides an artificial disc with a mobile center of rotation which more closely mimics anatomical motion than the known ball and socket type artificial discs.

The shock absorbing core includes one or more spring washers or disc springs between upper and lower surfaces of the core to allow the upper and lower surfaces to move resiliently toward and away from each other. This allows the core to absorb forces applied to it by the vertebrae. The components of the shock absorbing core including the disc springs are formed of rigid materials having high durability and biocompatibility.

Intervertebral discs must fit into the space between adjacent vertebrae. This intervertebral space is on the order of 1-2 cm in the lumbar region and half of that in the cervical region. This restricted space leaves very little room to accommodate a spring element to provide desired compliance to an artificial disc design. Compliant and highly elastic materials could be used to create a spring element in this small space (e.g. elastomers with large recoverable strains of approximately 25%). However, these elastomers can break down after long exposure in the body and lack the durability of rigid materials. An all metal design utilizing disc springs or spring washers provides the advantage of long term stability in the body. The yield strains of metals are also nearly 2 orders of magnitude lower than those of elastomeric materials. Spring washers and disc springs are particularly suited for this application to provide a compliant element that can accommodate a relatively large load within a very small height.

The shock absorbing cores described herein can be used with many artificial disc designs and with different approaches to the intervertebral disc space including anterior, lateral, posterior and posterior lateral approaches. Although various embodiments of such an artificial disc are shown in the figures and described further below, the general principles of these embodiments, namely providing a resilient core with a force absorbing design, may be applied to any of a number of other disc designs including other mobile core designs as well as ball and socket designs. In some embodiments, the shock absorbing core can be used with an expandable intervertebral prosthesis, as described in U.S. application Ser. No. 11/787,110, entitled “Posterior Spinal Device and Method”, filed Apr. 12, 2007, Publication No. 2007-0282449, the full disclosure of which is incorporated herein by reference.

Disc springs as used in the shock absorbing cores of the present invention are particularly well suited for carrying large loads in very small spaces and for providing small deflections and extremely long fatigue life. Disc springs come in a variety of configurations and can be stacked in different manners to tailor the loads and deflections to a particular application. Disc springs include both spring washers with a central hole, and disc springs without a central hole. In addition, the spring washers or discs may be slotted, tapered, split, or contoured in a variety of ways.

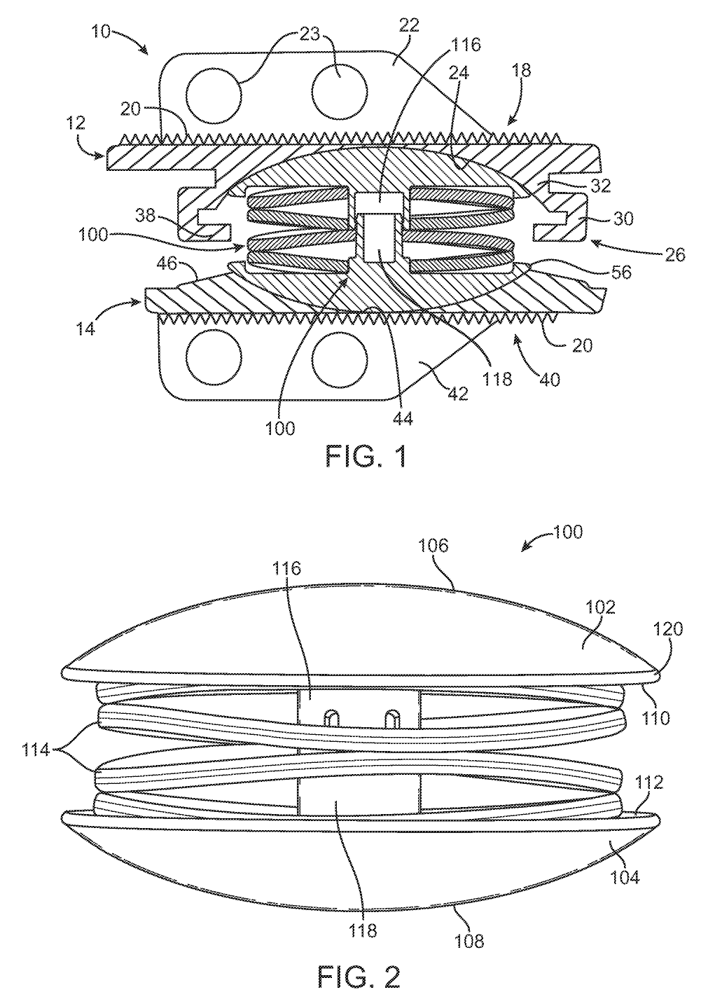

FIG. 1 shows an artificial disc 10 having a shock absorbing core 100. The disc 10 for intervertebral insertion between two adjacent spinal vertebrae (not shown) includes an upper plate 12, a lower plate 14 and the movable shock absorbing core 100 located between the plates. The cross section of FIG. 1 is taken along a line in the anterior-posterior direction of the implanted disc.

The upper plate 12 includes an outer surface 18 and an inner surface 24 and may be constructed from any suitable metal, alloy or combination of metals or alloys, such as but not limited to cobalt chrome molybdenum alloys, titanium (such as grade 5 titanium), stainless steel and/or the like. In one embodiment, typically used in the lumbar spine, the upper plate 12 is constructed of cobalt chrome molybdenum, and the outer surface 18 is treated with aluminum oxide blasting followed by a titanium plasma spray. In another embodiment, typically used in the cervical spine, the upper plate 12 is constructed of titanium, the inner surface 24 is coated with titanium nitride, and the outer surface 18 is treated with aluminum oxide blasting. An alternative cervical spine embodiment includes no coating on the inner surface 24. In other cervical and lumbar disc embodiments, any other suitable materials or combinations thereof, such as PEEK (polyetheretherketone) or combinations of metals, non-metals, and coatings may be used. In some embodiments, it may be useful to couple two materials together to form the inner surface 24 and the outer surface 18. For example, the upper plate 12 may be made of an MRI-compatible material, such as titanium, but may include a harder material, such as cobalt chrome molybdenum, for the inner surface 24. In another embodiment, upper plate 12 may comprise a metal, and inner surface 24 may comprise a hard ceramic material. Any suitable technique may be used to couple materials together, such as snap fitting, slip fitting, lamination, interference fitting, use of adhesives, welding and/or the like. Any other suitable combination of materials and coatings may be employed in various embodiments of the invention.

In some embodiments, the outer surface 18 is planar. Oftentimes, the outer surface 18 will include one or more surface features and/or materials including serrations, fins, coatings, teeth or threaded fasteners to enhance attachment of the prosthesis 10 to vertebral bone. For example, the outer surface 18 may be machined to have serrations 20 or other surface features for promoting adhesion of the upper plate 12 to a vertebra. In the embodiment shown, the serrations 20 are pyramid shaped serrations extending in mutually orthogonal directions, but other geometries would also be useful. Additionally, the outer surface 18 may be provided with a rough microfinish formed by blasting with aluminum oxide microparticles or the like. In some embodiments, the outer surface may also be titanium plasma sprayed to further enhance attachment of the outer surface 18 to vertebral bone.

The outer surface 18 may also carry one or more upstanding, vertical fins 22 extending in an anterior-posterior direction. In one embodiment, the fin 22 is pierced by transverse holes 23 for bone ingrowth. In alternative embodiments, the fin 22 may be rotated away from the anterior-posterior axis, such as in a lateral-lateral orientation, a posterolateral-anterolateral orientation, or the like depending on the direction of insertion of the disc. In some embodiments, the fin 22 may extend from the surface 18 at an angle other than 90°. Furthermore, multiple fins 22 may be attached to the surface 18 and/or the fin 22 may have any other suitable configuration, in various embodiments. In some embodiments, such as discs 10 for cervical insertion, the fins 22, 42 may be omitted altogether.

The inner, spherically curved concave surface 24 provides a bearing surface for the shock absorbing core 100. At the outer edge of the curved surface 24, the upper plate 12 carries peripheral restraining structure comprising an integral ring structure 26 including an inwardly directed rib or flange 38. The flange 38 forms part of a U-shaped member 30 joined to the major part of the plate by an annular web 32. Other types of restraining structures can also be used to retain the core 100 between the plates 12, 14 including discontinuous annular ring structures and other types of mating projections and recesses.

The lower plate 14 is similar to the upper plate 12 except for the absence of the peripheral restraining structure 26. Thus, the lower plate 14 has an outer surface 40 which is planar, serrated and microfinished like the outer surface 18 of the upper plate 12. The lower plate 14 optionally carries one or more fins 42 similar to the fin 22 of the upper plate. The inner surface 44 of the lower plate 14 is concavely, spherically curved with a radius of curvature matching that of the shock absorbing core 100 to provide a bearing surface for the core. Once again, the inner surface 44 may be provided with a titanium nitride or other finish. Although a disc with two inner spherically curved surfaces 24, 44 has been shown one or both of these bearing surfaces for receiving the core may take on another shape.

At the outer edge of the inner curved surface 44, the lower plate 14 is provided with an inclined ledge formation 46 which contacts the flange 38 of the upper plate to limit the range of motion of the plates. Alternatively, the lower plate 14 may include a peripheral restraining structure analogous to the peripheral restraining structure 26 on the upper plate 12. The retaining structure 26 has been shown on the upper plate 12, however, it should be understood that the retaining structure may alternatively be located on the lower plate 14.

The shock absorbing core 100 shown in FIGS. 1 and 2 and described herein includes upper and lower core members 102, 104 which are symmetrical about a central, equatorial plane. Although in other embodiments, the shock absorbing core 100 may be asymmetrical. The upper and lower core members 102, 104 include convexly curved outer surfaces 106, 108 configured to cooperate with the bearing surfaces 24, 44 of the upper and lower plates 12, 14. Opposite the convex outer surfaces 106, 108 of the core members are inner surfaces 110, 112 arranged to form contact surfaces for spring washers. Between the upper and lower core members 102, 104 are one or more spring washers 114, which in the embodiment of FIGS. 1 and 2 are four curved spring washers. The inner surfaces 110, 112 can be flat or contoured depending on the shape of the spring washers and the space needed for the one or more washers. For example, where more space is needed for the spring washers, the inner surfaces 110, 112 may be recessed within the upper and/or lower core members to form a chamber to accommodate at least a portion of the one or more washers 114.

When the plates 12, 14 and shock absorbing core 100 are assembled and in the orientation seen in FIG. 1, a rim or lip 120 of the upper core member 102 fits above the flange 38 on the upper plate 12 so that as the core moves within the disc 10 it is retained by the flange 38. The flange 38 prevents separation of the core 100 from the plates. In other words, the cooperation of the retaining formation 26 of the upper plate rim and the lip 120 ensures that the shock absorbing core 100 is held captive between the plates 10, 14 at all times during flexure of the disc 10.

Although the bearing surfaces 106, 108 of the core have been shown as spherical convex surfaces, these surfaces may alternatively take on many other shapes including flat, cylindrical, elliptical and concave. In each case at least a portion of the corresponding bearing surfaces 24, 44 of the plates correspond in shape to the bearing surfaces 106, 108 of the core. The upper and lower bearing surfaces 106, 108 can be the same or different shapes.

The outer diameter of the lips 120 on the core are very slightly smaller than the diameter defined by the inner edge of the flange 38 to allow the core to be placed into the opening in the top plate 12. In another embodiment, the shock absorbing core 100 is movably fitted into the upper plate 12 via an interference fit. To form such an interference fit with a metal core 100 and metal plate 12, any suitable techniques may be used. For example, the plate 12 may be heated so that it expands, and the core 100 may be dropped into the plate 12 in the expanded state. When the plate 12 cools and contracts, the interference fit is created. In another embodiment, the upper plate 12 may be formed around the shock absorbing core 100. Alternatively, the shock absorbing core 100 and upper plate 12 may include complementary threads, which allow the shock absorbing core to be screwed into the upper plate 12, where it can then freely move.

In an alternative embodiment, the continuous annular flange 38 may be replaced by a retaining formation comprising a number of flange segments which are spaced apart circumferentially. In yet another embodiment, the retaining formation(s) can be carried by the lower plate 14 instead of the upper plate 12, i.e. the plates are reversed. In some embodiments, the upper (or lower) plate is formed with an inwardly facing groove, or circumferentially spaced groove segments, at the edge of its inner, curved surface, and the outer periphery of the core 100 is formed with an outwardly facing flange or with circumferentially spaced flange segments.

In use, the disc 10 is surgically implanted between adjacent spinal vertebrae in place of a damaged disc which has been removed by a known discectomy procedure. The adjacent vertebrae are forcibly separated from one another with known distraction tools to provide the necessary space for insertion. The disc 10 is typically, though not necessarily, advanced toward the disc space from an anterolateral or anterior approach and is inserted in a posterior direction—i.e., from anterior to posterior. The disc 10 is inserted into place between the vertebrae with the fins 22, 42 of the top and bottom plates 12, 14 entering slots cut in the opposing vertebral surfaces to receive them. During and/or after insertion, the vertebrae, facets, adjacent ligaments and soft tissues are allowed to move together to hold the disc in place. The serrated and microfinished surfaces 18, 40 of the plates 12, 14 locate against the opposing vertebrae. The serrations 20 and fins 22, 42 provide initial stability and fixation for the disc 10. With passage of time, enhanced by the titanium or other surface coating, firm connection between the plates and the vertebrae will be achieved as bone tissue grows over the serrated surface. Bone tissue growth will also take place about the fins 22, 40 and through the transverse holes 23 therein, further enhancing the connection which is achieved.

In the assembled disc 10, the complementary and cooperating spherical surfaces of the plates 12, 14 and shock absorbing core 100 allow the plates to slide or articulate over the core through a fairly large range of angles and in all directions or degrees of freedom, including rotation about the central axis. FIG. 1 shows the disc 10 with the plates 12 and 14 and shock absorbing core 100 aligned vertically with one another.

### Curved Spring Washers

Referring now to FIGS. 1 and 2, a shock absorbing core 100 is shown in which the core includes a plurality of curved spring washers 114. The curved spring washers 114 can be as simple as flat disc shaped washers which are bent in a single direction. The elasticity of the curved spring washers 114 is a result of the resistance of the washers to flattening. The embodiment of FIGS. 1 and 2 shows four curved washers 114 arranged in series (with alternating directions of curvature). The washers 114 are preferably oriented at a fixed orientation so that the peaks of the adjacent washers remain in contact with each other to achieve consistent deflections. When the washers 114 are compressed during loading, the diameter of the washers expand.

As shown in FIG. 1, the upper and lower disc members 102, 104 include telescoping central posts 116, 118 which snap together with a retention feature which will be described in further detail below. The washers 114 may be keyed onto the central posts 116, 118 to prevent rotation of the washers from their desired orientation. The four curved spring washers 114 are placed over the central telescoping posts 116, 118 before the retention features are snapped together. A total deflection of the compliant core 100 is determined by the curvature of the curved washers 114. The inner surfaces 110, 112 of the upper and lower disc members 102, 104 are preferably formed of a hard material to form a bearing surface which is resistant to wear by the washers which may tend to dig into the bearing surface due to their shape. Although the curved washers 114 are illustrated as circular washers, they can be formed in other shapes, such as rectangular washers or circular washers with truncated or flattened ends.

### Flat Spring Washers

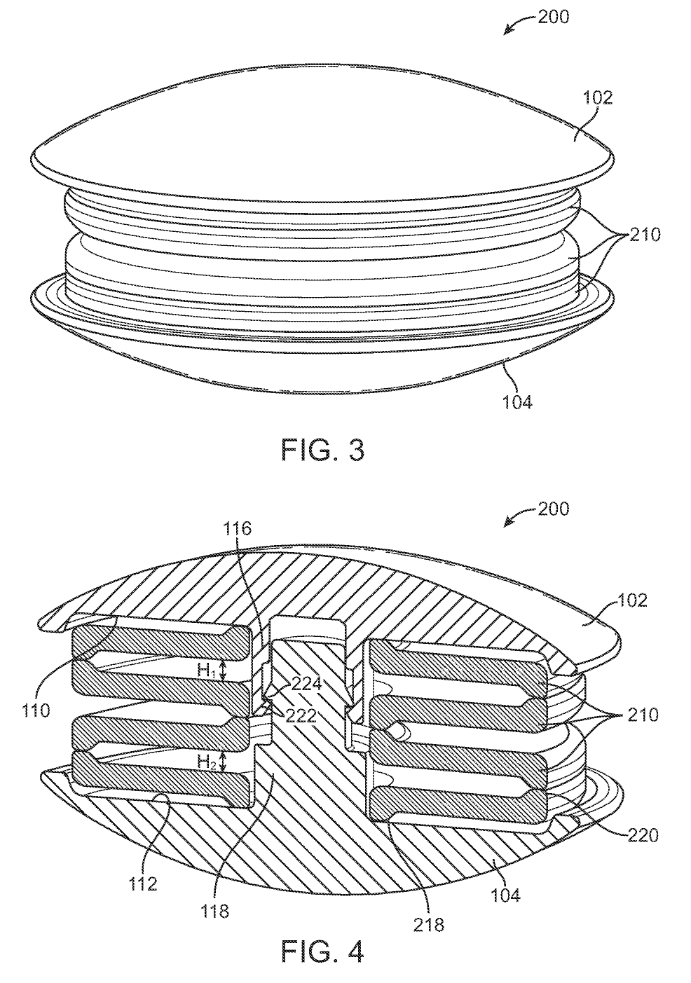

FIGS. 3-8 illustrate variations of a shock absorbing core 200 with flat spring washers 210 which are deflected out of their flat configuration into domed or conical shapes by application of a load to the core. Flat spring washers 210 can be flat or essentially flat with one or more ribs, rings, lips or other features for transferring forces to and from the flat washers. The resilience of the flat spring washers 210 is a result of resistance of the spring to moving out of the flat configuration. The flat spring washers 210 may include curves and bends, ribs, or slots which will change the stiffness of the springs.

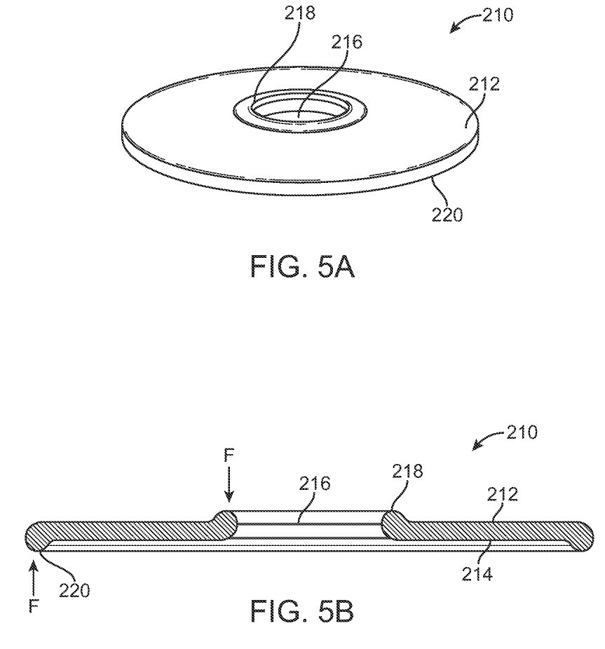

FIGS. 5A and 5B illustrate a single flat washer 210 having a substantially flat top surface 212 and a substantially flat bottom surface 214. The top and bottom surfaces 212, 214 can have a somewhat contoured surface to distribute strain within the washer 210. The washer 210 has a central hole 216 and an inner lip 218 surrounding the hole. The inner lip 218 may be spaced from the hole or the hole may be omitted all together in some designs providing a disc spring. An outer periphery of the washer 210 has an outer lip 220 which extends from the flat portion of the washer 210 in an axial direction opposite of the inner lip 218. Application of a force to the spring washer 210 at the lip portions 218, 220 in the directions of the arrows F shown in FIG. 5B causes the spring washer to deform out of its original substantially flat plane. Removal of the force F allows the spring washer 210 to return to its original shape.

The flat spring washers 210 are shown in FIGS. 3-8 have rims or lips 218, 220 for transferring forces to the washers and for spacing the washers from adjacent structures in the core to allow the washers space to deform. The rims or lips 218, 220 may be replaced with other structures, such as discontinuous tab structures. Although the ribs 218, 220 for transferring forces to the washers 210 are shown on the washers themselves, these ribs can alternatively or additionally be formed on the inner contact surfaces 110, 112 of the upper and lower core members 102, 104. The rims 218, 220 may be replaced with other force transferring structures on the washers or on adjacent structures. For example, in a core with a single flat spring washer, the washer may be flat and the upper and lower core members 102, 104 may be provided with projecting rims one near the inner edge of the washer and one near the outer edge of the washer.

The compliant core 200 shown in FIG. 4 has a locking retention feature in the form of the telescoping central posts 116, 118 with mating snap lock projections 222, 224. When the telescoping posts 116, 118 are in the locked position, the washers 210 may be arranged in 1) a tightly nested configuration without any space for free movement, 2) in a loosely stacked arrangement with free play between the washers within the core, or 3) a preloaded configuration in which the washers are tightly packed and slightly deformed. A preloaded configuration provides the advantage of a compliant disc which can be designed to have the same height when the patient is at rest and at stance. The preloading minimizes or eliminates compression of the core while the patient is standing up and maintains all of the potential core deflection for the impact loads after standing that the disc is designed to absorb. A preloaded spring configuration is described in U.S. Provisional Patent Application Ser. No. 61/023,536 filed on Jan. 25, 2008, which is incorporated herein by reference in its entirety.

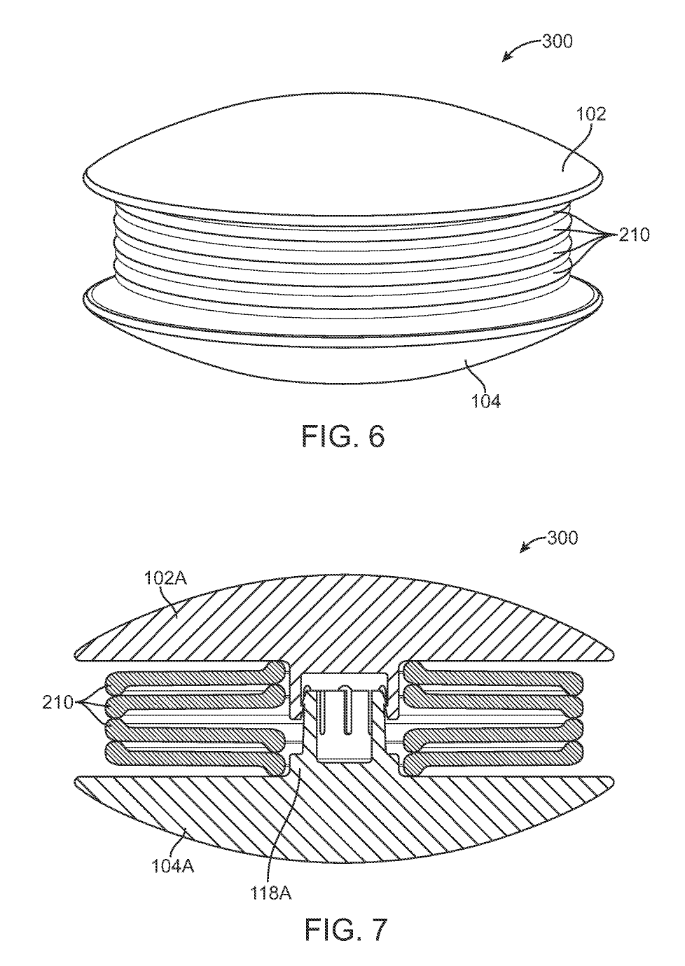

An amount of maximum total deflection provided by a core such as the core 200 in FIG. 4 is measured as a total of the heights H₁ and H₂ between the washers. When the washers 210 are placed in series as in FIG. 4, the four washers provide four times the deflection of a single washer for the same stress. Washers may also be arranged in parallel as will be shown in FIGS. 6 and 7 to increase the load bearing capacity.

FIGS. 6 and 7 illustrate a shock absorbing core 300 having upper and lower core members 102A and 104A and four flat spring washers 210. The upper and lower core members 102A, 104A of the core 300 have flat inner surfaces and modified central posts from the core 200 of FIG. 4, however, the core works in the same manner. The modified central post of the shock absorbing core 300 includes a hollow slotted lower post 118A. The flat washers 210 as seen in FIG. 7 are arranged with the top two washers in a parallel arrangement and the bottom two washers in a parallel arrangement to increase the load bearing capacity of the core. The upper and lower pairs of washers 210 are arranged in series.

### Cone Spring Washers

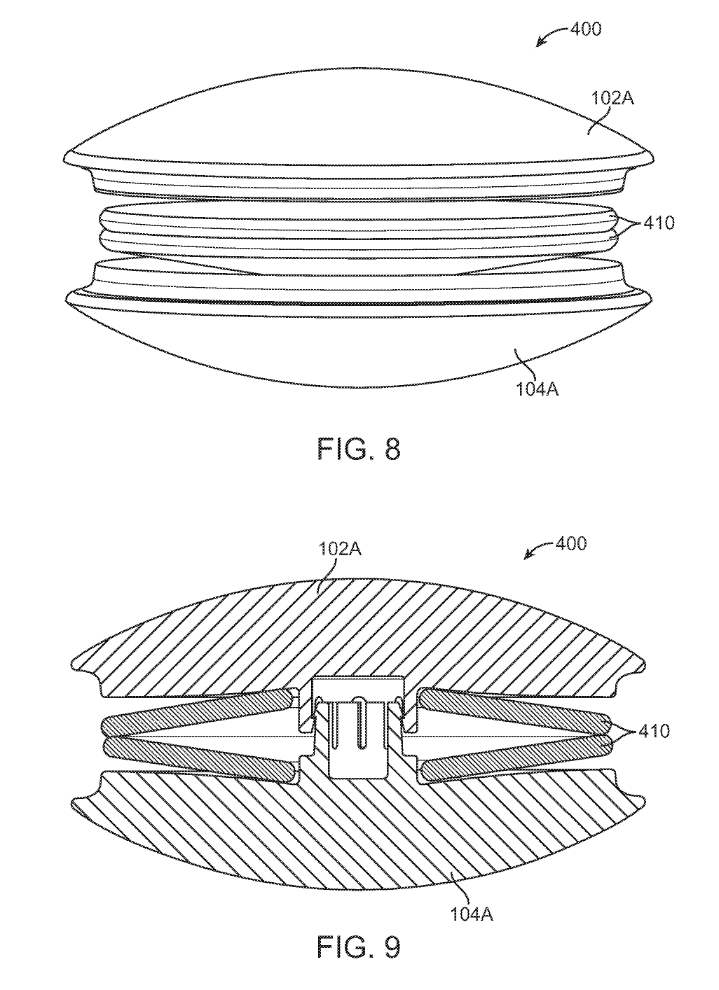

FIGS. 8 and 9 show a shock absorbing core 400 having two cone shaped spring washers 410. The cone shaped washers 410 are arranged between the upper and lower core members 102A, 104A with the narrow ends of the cones in contact with the bearing surfaces 110A, 112A of the core members and the wide ends of the two cones in contact with each other. Cone shaped washers function by flattening in response to axial forces applied to the cone. The use of cone shaped washers as well as the other washers and disc springs described herein are particularly advantageous where space is limited and high force is required.

The cone shaped washers 410 can be arranged in parallel and/or series stacks and any number of washers can be used.

### Wave Spring Washers

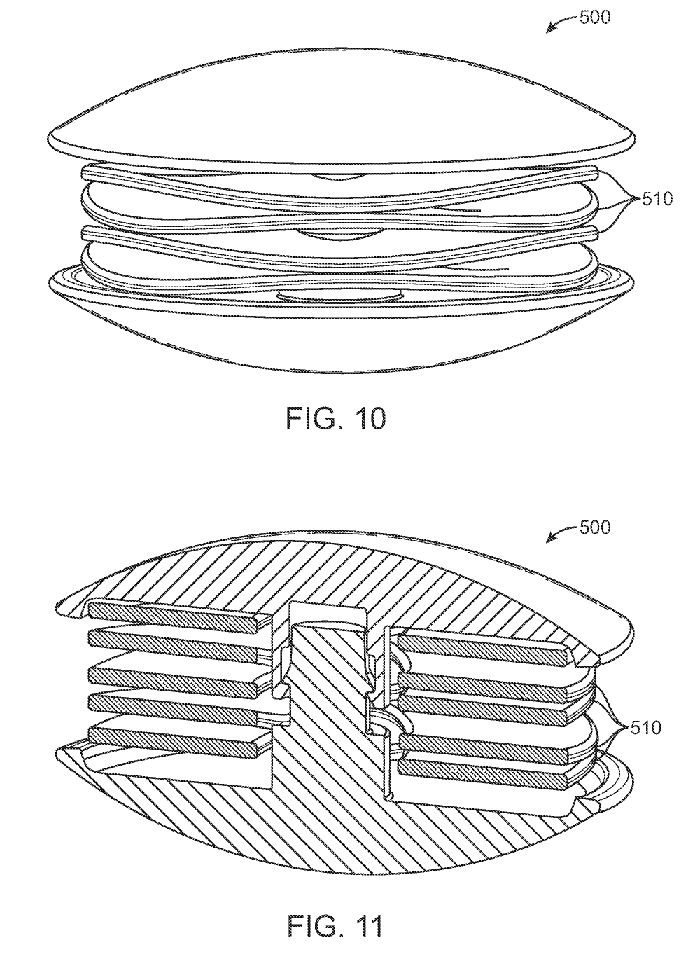

FIGS. 10-12 illustrate a shock absorbing core 500 having a plurality of wave spring washers 510. The elasticity of the wave springs 510 is a result of their resistance to flattening. Although the wave springs 510 are shown with holes in the middle, they can also be formed without the central hole and function in a similar manner as wave disc springs. The core 500 of FIG. 11 has 5 wave springs 510 arranged in series to provide 5 times the deflection of a single wave spring with the same stress.

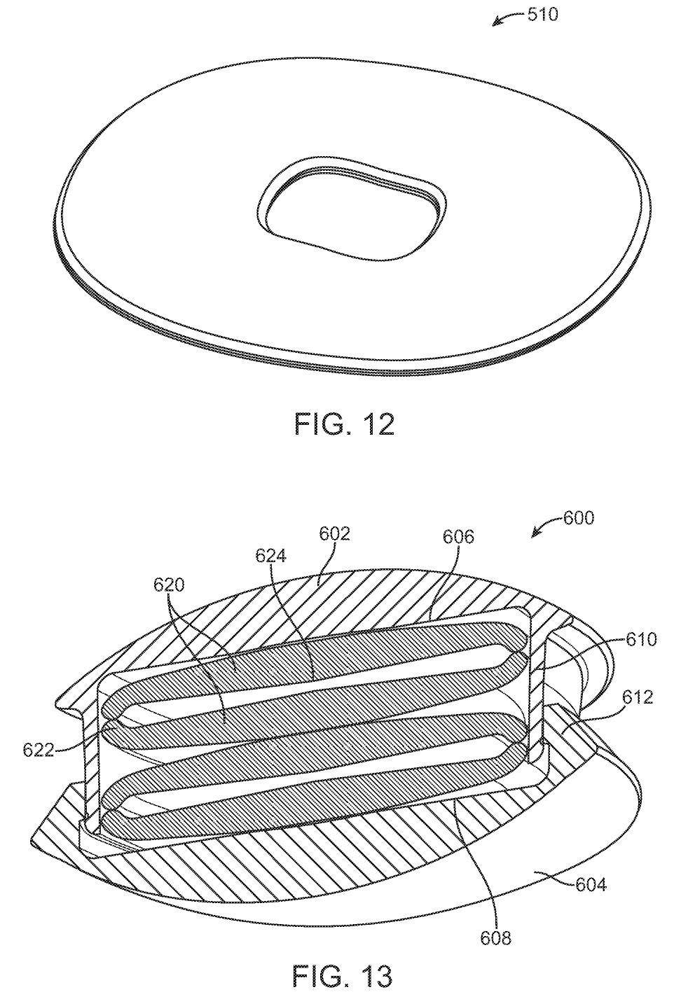

A single wave spring washer 510 is shown in FIG. 12. In a preferred embodiment, the wave springs 510 are loaded around the central post of the core 500 with the springs registered in a particular angular position so that the peak of each spring is directly opposed to the adjacent peak of the next spring. The registration may be accomplished in a number of ways, such as, by providing a protrusion on the core that engages with a notch in the wave spring washers 510 or by providing a non circular central post and corresponding non-circular central holes in the wave springs. The wave springs 510 will act in series when the peaks are aligned with adjacent peaks or can be assembled to act in parallel by stacking them in an aligned mating orientation.

### Flat Disc Springs

The core 600 of FIG. 13 includes flat disc springs 620 which differ from the flat spring washers of FIGS. 3-8 in that the central hole can be omitted. The flat disc springs 620 function by moving out of their substantially flat configuration to a more dished configuration in response to applied forces. The springs 620 are stacked within a cavity formed by upper and lower core members 602, 604 and are retained in this stacked configuration by a retaining feature on the perimeter of the core. The retaining feature is formed by a pair of snap locking annular walls 610, 612 which permanently trap the springs 620 in the cavity between the core members 602, 604.

The springs 620 shown in FIG. 13 have an annular lip 622 and a central thicker portion 624 which function as bearing surfaces for the springs 620. However other alternative bearing surfaces on either the springs 620 themselves or on the upper and lower core members 602, 604 can be used. Although the disc springs shown in FIG. 13 are substantially flat, other disc spring variations can include curved springs, wave springs, conical springs, and dome shaped springs.

The core designs described above can all be modified to provide more stability in shear by modification of the central posts or other retention feature. Stiffness can be increased or decreased as necessary by increasing or decreasing the thickness of the spring washers or disc springs. Compliance can be increased or decreased by modifying the spacing between the spring washers or disc springs.

### Split Washer Spring

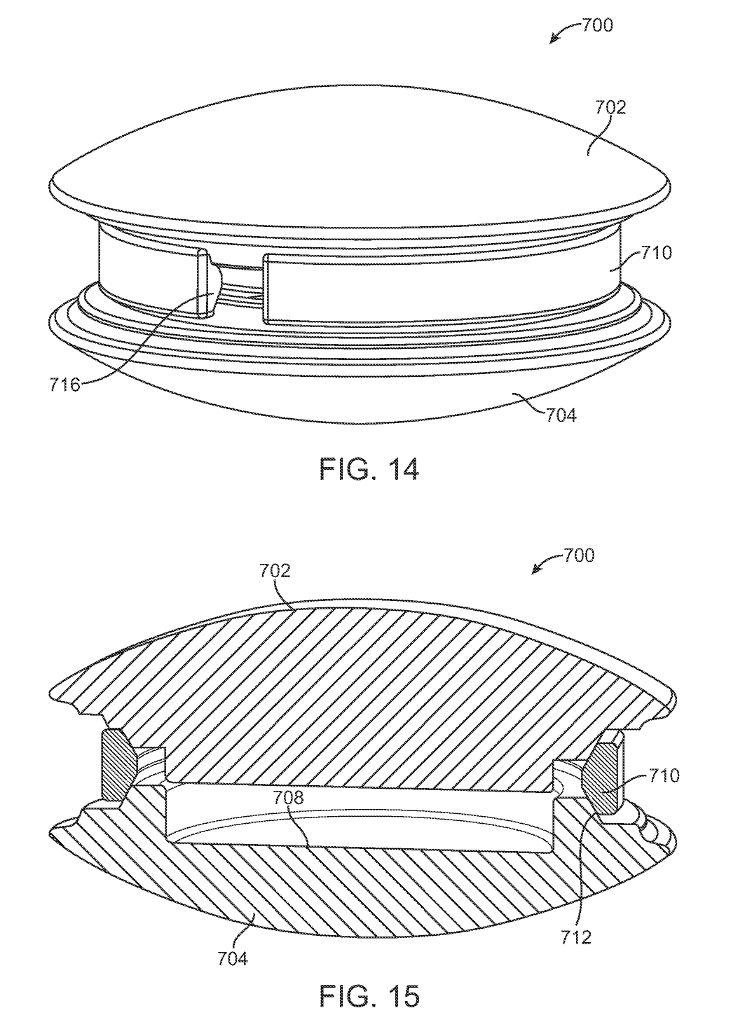

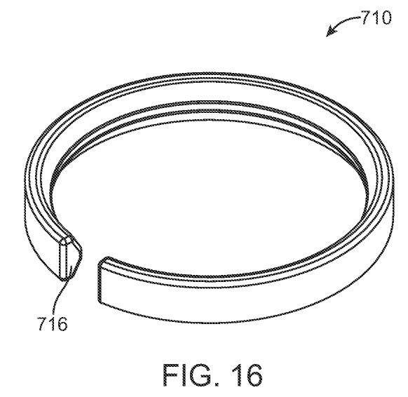

FIGS. 14-16 illustrate an alternative embodiment of a shock absorbing core 700 in which compliance is provided by a split washer spring 710. The core 700 includes upper and lower core members 702, 704 which are configured to engage one another with a telescoping arrangement. The spilt washer spring 710 is shown in FIG. 16 and has tapering inner contact surfaces. The upper and lower cores members 702, 704 are also provided with angled annular surfaces 712 which serve as bearing surfaces for the split washer spring 710. In operation, the split washer spring 710 provides compliance by opening at the split 716 and expanding in diameter in response to the application of an axial load to the core 700. The core 700 may also be provided with a snap lock retention feature (not shown) which locks the upper and lower core members 702, 704 together as in the previous embodiments.

### Split Spring Coil Washer

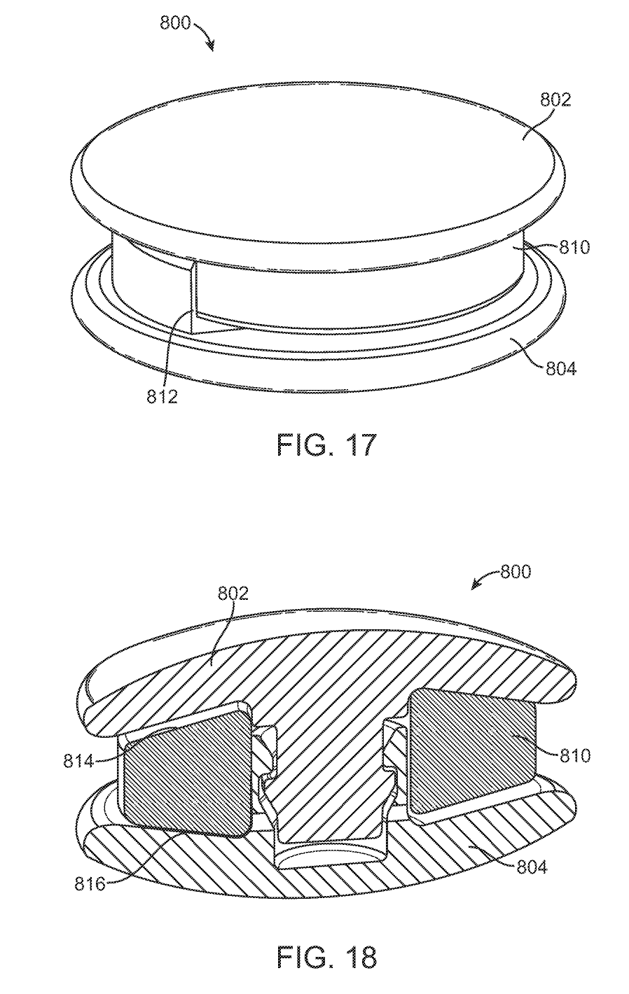

Another version of a core with a split spring washer is shown in FIGS. 17 and 18. A shock absorbing core 800 includes upper and lower core members 802, 804 and a single split spring washer 810. This single split spring washer embodiment is particularly useful in smaller applications, such as the cervical application where the total height of the core may be on the order of 5 mm. As shown in FIG. 17, the split washer 810 is bent in the manner of a common lock washer to form what is essentially just less than one turn of a coil spring. This results in an offset of the ends of the split spring washer at the location of the split 812 in the washer. In operation, as the upper and lower core members 802, 804 move toward each other while the core 800 is compressed, the split spring washer 810 deforms from the offset or coil configuration to a flat configuration. Upper and lower surfaces 814 and 816 of the washer 810 and corresponding surfaces of the upper and lower core members 802, 804 are tapered to assist in retaining the washer within the core 800. However, other shapes of these surfaces may also be employed.

The tapered shape of the split spring washer 810 in cross section and corresponding shape of the upper and lower core members provides a safety feature by trapping the spring in the case where the spring has fractured.

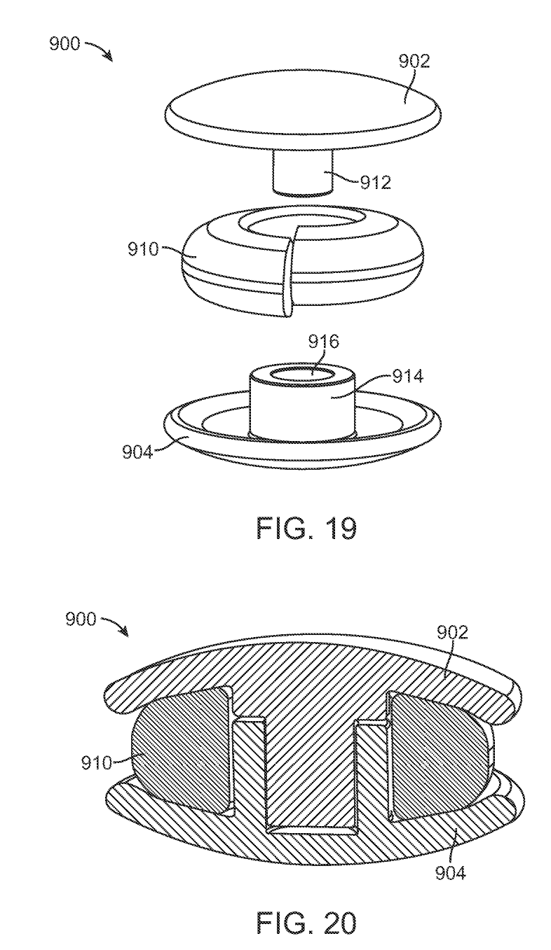

FIGS. 19 and 20 illustrate another embodiment of a core 900 having a split spring washer 910 in the form of a coil. The core 900 functions substantially like the core 800 of FIG. 17. It is noted that FIG. 20 shows the core 900 in the completely compressed or bottomed out configuration. As shown in FIGS. 19 and 20, an upper core member 902 includes a solid central post 912 while a lower core member 904 has a central post 914 with a central bore 916. The post 912 of the upper core member 902 fits within the central post 914 of the lower core member 904 in a telescoping and non-locking arrangement. The upper and lower core members 902, 904 are prevented from separating during and after implantation by the inwardly directed force between the plates provided first by an implantation tool and after implantation by the anatomy of the spine. In addition, some additional temporary connection method may be provided for holding to core together. For example, a snug friction fit between the posts 912, 914 or biodegradable adhesive may be used.

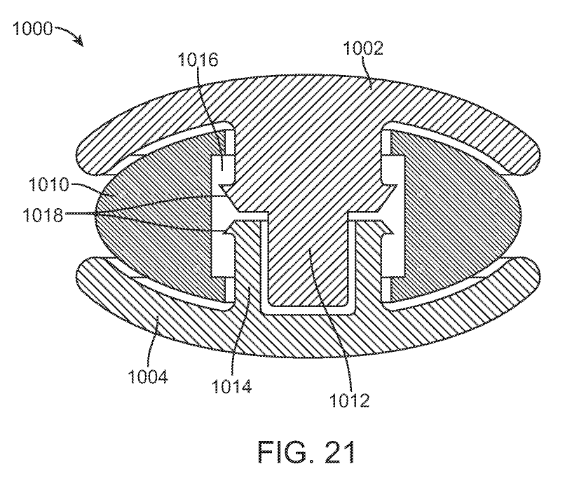

FIG. 21 shows another alternative embodiment of a core 1000 with a split spring coil washer 1010. The core 1000 has a telescoping upper and lower core member configuration with an upper core member post 1012 telescoping within a lower core member post 1014. The core 1000 is provided with a retention feature to prevent the upper and lower members 1002 and 1004 of the core from separating. The retention feature includes projections 1018 on both the posts 1012, 1014 which mate with a channel 1016 in the spring 1010. The spring coil 1010 expands during assembly so that the upper and lower core members 1002, 1004 snap into place.

Preferably the shock absorbing cores including the upper and lower core members and the spring washers or disc springs are made of metal such as titanium, cobalt chromium alloy, stainless steel, NiTi or a combination thereof. These materials provide a high hardness surface for the upper and lower surfaces of the cores which improve performance and prevent particulate generation. These materials also can be designed to provide spring washers which are deformable in the elastic region of the stress/strain curve and will not plastically deform during compression. Typically when using metallic cores and springs the spring washers and upper and lower core members are formed of similar materials to avoid galvanic corrosion. Galvanic corrosion refers to the damage induced when two dissimilar metals are coupled. However, when a polymer is used for the upper and lower core members, such as the highly stable and strong polymer polyetheretherketone (PEEK), a metallic spring element can be used without the concern of galvanic corrosion. Thus, a PEEK core may be made with a nickel titanium or other superelastic material used as the spring washer.

The materials, shape, size, number, arrangement (series or parallel stacking) and other features of the spring washers can be varied to provide tailored compliance properties of all different kinds of spring washers and disc springs. The spring washers and disc springs can also be modified to provide tailored behaviors, such as non linear spring behaviors providing progressively stiffer behavior upon larger compression.

The cores according to present invention can be designed to provide preferential deflection in one direction over another direction by providing asymmetrical springs or asymmetrical upper and lower core members.

The shock absorbing cores preferably have a predesigned hard stop so that the maximum amount of deflection of the springs is limited to a predetermined amount according to the application.

Preloading of the springs in the present invention can be performed by placing the springs between the upper and lower core members in a configuration in which the cores are unloaded and loading the cores while snapping the retention features together. Preloading provides, among other advantages, the advantage that the entire deflection available in the core is available for shock absorption at the standing position. Preloading can substantially eliminate the compression of the core that would otherwise occur during a change from lying to standing position.

In each of the shock absorbing cores described herein, the multiple parts within the cores are designed for minimal or no motion between contacting parts within the cores to prevent particulate generation. However, since the cores are made entirely of hard materials such as metals, some minimal rubbing contact may be accommodated.

In one embodiment of the present invention, for a cervical application, the maximum deformation of the shock absorbing disc is about 0.1 to about 1.0 mm, and is preferably about 0.2 to about 0.8 mm. For a lumbar application, the maximum deformation of the shock absorbing disc is about 0.2 to about 2.0 mm, and is preferably about 0.4 to about 1.5 mm.

The cores are configured to begin deflection at a load of about 500-3000N for lumbar and about 50-300N for cervical applications. The cores are designed to bottom out, or complete maximum deflection at about 1000-5000N for lumbar and about 100-500N for cervical.

The shock absorbing cores can be provided with differing heights and differing resiliencies, for different patients or applications.

Although the shock absorbing core has been illustrated with respect to a movable core design of an artificial disc, the shock absorbing core can also be incorporated into one of the parts of a two piece ball and socket motion artificial disc. In the case of a ball and socket design the shock absorbing portion can be incorporated into the ball or the socket portion of the artificial disc.

In many embodiments, the shock absorbing core can be compressed with an instrument during insertion to allow for a lower profile during insertion. The insertion instrument may attach to the upper and lower plates of the artificial disc with the core in a compressed configuration.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. An artificial intervertebral disc comprising:
upper and lower supports, each support comprising,
an outer surface which engages a vertebra, and
an inner bearing surface;
a core positioned between the upper and lower supports and movable with respect to the upper and lower supports, the core comprising,
upper and lower core members configured to engage the inner bearing surfaces of the upper and lower support plates, and
at least one spring washer in the core to provide compliance to the core which allows the upper and lower core members to move resiliently toward and away from each other.

2. The disc of claim 1, wherein the washer is a wave spring washer.

3. The disc of claim 2, wherein the core includes a plurality of wave spring washers.

4. The disc of claim 1, wherein the washer is a cone shaped washer.

5. The disc of claim 4, wherein the core includes a plurality of cone shaped washers.

6. The disc of claim 1, wherein the washer is a flat washer having one or more lips and the flat washer is deformed from a substantially planar configuration to a non-planar configuration to provide compliance to the core.

7. The disc of claim 6, wherein the core includes a plurality of flat washers arranged in series.

8. The disc of claim 6, wherein the core includes a plurality of flat washers arranged in parallel.

9. The disc of claim 6, wherein the flat washer is a ring shaped washer with a first lip at an exterior edge and a second lip at an interior edge and wherein forces applied to the washer to deform the washer are applied through the first and second lips.

10. The disc of claim 1, wherein the upper and lower core members lock together with a retention feature that allows relative motion of the members and prevents the members from being separated during use.

11. The disc of claim 10, wherein the retention feature is located at a center of the core and within a hole in the washer to prevent the washer from being removed from the core.

12. The disc of claim 10, wherein the retention feature includes a snap locking barb.

13. The disc of claim 10, wherein the retention feature is located at a periphery of the core surrounding the washer.

14. The disc of claim 13, wherein the peripheral retention feature is in the form of an annular ring with a snap lock feature which prevents the washer from being separated from the core during use.

15. The disc of claim 1, wherein the washer is a curved washer.

16. The disc of claim 15, wherein the core includes a plurality of curved washers.

17. The disc of claim 1, wherein the core has a maximum compression of about 2 mm or less.

18. The disc of claim 1, wherein the core has a maximum compression of about 1 mm or less.

19. The disc of claim 1, wherein the core has a minimum compression of about 0.05 mm.

20. The disc of claim 1, wherein the upper and lower core members and the washers are metallic.

21. The disc of claim 1, wherein the upper and lower core members and the washers comprise a cobalt chromium alloy, stainless steel, titanium, or NiTi.

22. The disc of claim 1, wherein the upper and lower core members have convexly curved surfaces configured to engage the inner bearing surfaces of the upper and lower plates.

23. The disc of claim 1, wherein the core is movable between the upper and lower supports after implantation of the disc in a patient.

24. A method of assembling a compliant mobile core for an artificial intervertebral disc, the method comprising:
providing upper and lower core members;
positioning at least one disc spring between the upper and lower core members in an arrangement which allows the upper and lower core members to move resiliently toward and away from each other; and
locking the upper and lower core members together in a manner which traps the at least one disc spring in place between the upper and lower core members.

25. The method of claim 24, wherein the disc spring is a wave washer.

26. The method of claim 24, wherein the disc spring is a cone shaped washer.

27. The method of claim 24, wherein the disc spring is a flat washer having one or more lips.

28. The method of claim 24, wherein the disc spring is a curved washer.

29. The method of claim 24, wherein the at least one disc spring includes a plurality of disc springs stacked between the upper and lower core members.

30. The method of claim 24, wherein the step of locking the upper and lower core members together includes a snap lock between the members.

31. The method of claim 24, wherein the disc spring is a split spring washer.

32. A mobile core for an artificial intervertebral disc, the core comprising:
upper and lower core members each having an outer bearing surface and an inner surface;
at least one spring washer positioned between the inner surfaces of the upper and lower core members to allow the upper and lower core members to move resiliently toward and away from each other; and
a retention feature on the upper and lower core members which prevents the spring washer from being removed from the core during use.

33. The core of claim 32, wherein the spring washer is a one of a wave spring washer, a cone shaped washer, a flat washer, a split washer or a curved washer.

34. The core of claim 33, wherein the core includes a plurality spring washers.

35. The core of claim 32, wherein the washer is a flat washer having one or more lips and the flat washer is deformed from a substantially planar configuration to a non-planar configuration to provide compliance to the core.

36. The core of claim 32, wherein the core includes a plurality of spring washers arranged in series.

37. The core of claim 32, wherein the core includes a plurality of spring washers arranged in parallel.

38. The core of claim 31, wherein the core has a maximum compression of about 2 mm or less.

39. The core of claim 32, wherein the core has a maximum compression of about 1 mm or less.

40. The core of claim 32, wherein the core reaches a maximum deflection at between about 200N and about 2000N.

41. An artificial intervertebral disc comprising:
upper and lower supports, each support comprising,
an outer surface which engages a vertebra, and
an inner bearing surface; and
a core positioned between the upper and lower supports and movable with respect to the upper and lower supports, the core comprising,
upper and lower core members configured to engage the inner bearing surfaces of the upper and lower support plates, and
at least one disc spring in the core to provide compliance to the core which allows the upper and lower core members to move resiliently toward and away from each other.

42. The disc of claim 41, wherein the core includes a plurality of disc springs.

43. The disc of claim 41, wherein the disc spring is a flat disc spring having one or more lips and the flat disc is deformed from a substantially planar configuration to a non-planar configuration to provide compliance to the core.

44. The disc of claim 41, wherein the disc spring is a washer.

45. The disc of claim 41, wherein the disc spring is a split spring washer.

46. The disc of claim 41, wherein the upper and lower core members lock together with a retention feature that allows relative motion of the members and prevents the members from being separated during use.

47. The disc of claim 46, wherein the retention feature is located at a periphery of the core surrounding the washer.

48. The disc of claim 41, wherein the upper and lower core members and the spring discs are formed of inelastic materials.

49. The disc of claim 41, wherein upper and lower core members are formed of polyetheretherketone (PEEK) and the spring discs are metallic.

50. The disc of claim 49, wherein the spring discs are formed of a nickel titanium alloy.
